(19) United States
(12) Patent Application Publication
(10) Pub. No.: US 2002/0177728 A1
(43) Pub. Date: Nov. 28, 2002

(54) Refining processes

(76) Inventors: Chris Nicholas Boudreaux, Westwego, LA (US); Alex Chiahuei Kuo, Luling, LA (US); Steven R. Luce, Boutte, LA (US); Timothy Jobe Mann, Luling, LA (US); Fungau Ho, Charleston, WV (US)

Correspondence Address: UNION CARBIDE CORPORATION, Law Department E205, 39 Old Ridgebury Road, Danbury, CT, 06817-0001, US

(21) Appl. No.: 09/817,081

(22) Filed: Mar. 26, 2001

(51) Int. Cl. C07C 69/74

(52) U.S. Cl. 560/218

(57) ABSTRACT

Crude C1-C8 alkyl (meth)acrylate streams, e.g., from the reaction of butanol and (meth)acrylic acid, are refined using a splitter distillation column to yield a C1-C8 alkyl (meth)acrylate- and heavies-containing bottoms fraction and an overhead fraction containing C1-C8 alkyl acetate, C1-C8 alcohol and C1-C8 alkyl (meth)acrylate. A stream comprising a water soluble organic solvent is introduced into the splitter distillation column in an amount sufficient to prevent the formation of water layers in the splitter distillation column and thereby reduce fouling in the splitter distillation column. The bottoms fraction is then subjected to separation, e.g., by distillation, to separate heavies from the bottoms fractions and to recover C1-C8 alkyl (meth)acrylate.

### BRIEF SUMMARY OF THE INVENTION

1. Technical Field

This invention pertains to processes for producing C1-C8 alkyl (meth)acrylates by the esterification of (meth)acrylic acid with C1-C8 alcohol and to processes for refining C1-C8 alkyl (meth)acrylates. In accordance with this invention, a splitter distillation column is used to make a separation between (i) lights, which include C1-C8 alkyl acetate, C1-C8 alcohol and lower boiling components and (ii) C1-C8 alkyl (meth)acrylate and heavies. The benefits of this invention include avoidance of column fouling, reduced inhibitor consumption, increased unit on stream time and reduced costs from splitter distillation column cleaning.

### BACKGROUND OF THE INVENTION

Alkyl (meth)acrylates are prepared by the equilibrium-limited reaction of (meth)acrylic acid and an alcohol. The reaction is conducted at elevated temperature in the presence of a catalyst. Typically, the coproduct, water, is removed during the course of the reaction to drive the reaction more toward the alkyl (meth)acrylate product. In conducting this reaction, impurities in the raw materials and side reactions generate by-products that must be removed from the alkyl (meth)acrylate.

Conventionally, the refining of the alkyl (meth)acrylate-containing reaction product is conducted by distillation. The purification is generally done in several distillation column sections. U.S. Pat. No. 4,012,439 discloses a process for producing n-butyl (meth)acrylate free from dibutyl ether. In the disclosed process, the esterification reaction product is sent to a first distillation column. A ternary mixture containing butyl (meth)acrylate, butanol and water is the distillate. This distillate is condensed and subjected to phase separation, and the organic phase is delivered to a second distillation column from which a ternary distillate of butyl (meth)acrylate, butanol and water is obtained. The base product from this second distillation column contains butanol and butyl (meth)acrylate and is passed to a third distillation column for the separation of butanol from butyl (meth)acrylate.

U.S. Pat. No. 4,280,010 discloses a process for making butyl (meth)acrylate in which the reaction overhead is passed to a reactor column with the distillate being a butyl (meth)acrylate, butanol and water azeotrope. A reflux is used in the reactor column. The remainder of the distillate is sent to a dehydration column and the organic product is further distilled to provide a butanol—butyl (meth)acrylate azeotrope being recycled to the reactor and butyl (meth)acrylate stream being obtained at the base of the column.

U.S. Pat. No. 4,814,493 discloses another technique for making butyl (meth)acrylate. The reaction overhead is passed to a finishing distillation column. The finishing distillation column provides a side stream of butyl (meth)acrylate product, a bottoms stream that is recycled to the reactor and an overhead stream that is passed to a butanol recovery distillation column. The distillate from the butanol recovery column is recycled to the reactor. This patent also describes the use of a heat treater which receives a portion of the liquid from the reactor. The heat treater apparently cracks components which can then be separated from heavies via distillation column and returned to the reactor.

Alkyl (meth)acrylates having high purity are sought. The process for making alkyl (meth)acrylates, and the sources of raw materials for those processes, result in troublesome separations. One impurity is a dialkylether which can have a normal boiling point within 5° to 7° C. of that of the alkyl (meth)acrylate. This impurity can impart odor to the alkyl (meth)acrylate. Another impurity is alkyl acetate which is the esterification product of acetic acid, an impurity in (meth)acrylic acid, and alkyl acetate can also impart odor to alkyl (meth)acrylate.

Processes for manufacturing and refining alkyl (meth)acrylates are sought which are economical, energy efficient and capable of providing alkyl (meth)acrylates of enhanced purity. Moreover, processes are sought that enable the use of (meth)acrylic acid containing acetic acid and reaction conditions which enable dialkyl ether to be formed, yet provide an alkyl (meth)acrylate product to be obtained of unexpectedly high purity.

### DISCLOSURE OF THE INVENTION

In accordance with the processes of this invention, a fluid stream, i.e., splitter feed stream, containing C1-C8 alkyl (meth)acrylate, C1-C8 alkyl acetate, and C1-C8 alcohol is separated in a splitter distillation column to provide an overhead fraction containing C1-C8 alkyl acetate and C1-C8 alcohol and a bottoms fraction containing C1-C8 alkyl (meth)acrylate and heavies. The overhead fraction typically contains C1-C8 alkyl (meth)acrylate in addition to C1-C8 alkyl acetate and C1-C8 alcohol. The bottoms fraction preferably contains less than about 0.2 weight percent of total C1-C8 alkyl acetate and less than about 10 percent of the total C1-C8 alkyl acetate in the fluid stream. The overhead fraction from the splitter column can be subjected to further distillation in the presence of water in a C1-C8 alcohol recovery column to separate C1-C8 alcohol and C1-C8 alkyl (meth)acrylate from lights containing C1-C8 alkyl acetate. The bottoms fraction from the splitter distillation column, comprising heavies and C1-C8 alkyl (meth)acrylate, can advantageously be rectified in a C1-C8 alkyl (meth)acrylate distillation column to separate heavies from the C1-C8 alkyl (meth)acrylate.

In the aspect of the invention relating to esterification processes, C1-C8 alcohol and (meth)acrylic acid are subjected, in at least one reaction zone, to esterification reaction conditions including the presence of an esterification catalyst, to produce C1-C8 alkyl (meth)acrylate and water as well as side products including C1-C8 alkyl acetate, heavies and di(C1-C8 alkyl) ether. A gaseous stream containing water, C1-C8 alcohol, (meth)acrylic acid, C1-C8 alkyl (meth)acrylate, C1-C8 alkyl acetate and heavies is withdrawn from the reaction zone and passed to a reactor distillation column. The overhead from the reactor distillation column contains water, C1-C8 alcohol, C1-C8 alkyl (meth)acrylate, C1-C8 alkyl acetate and heavies, and this overhead is condensed and subjected to liquid phase separation to remove most of the water, and the resulting organic phase is the liquid feed stream to the splitter distillation column as described above. The bottoms fraction from the reaction distillation column is rich in (meth)acrylic acid, at least a portion of which is recycled to at least one reaction zone.

The bottoms fraction from the splitter distillation column is advantageously rectified in a distillation column to separate heavies from the C1-C8 alkyl (meth)acrylate. Recovered C1-C8 alkyl (meth)acrylate has a purity of at least about 99.0 weight percent C1-C8 alkyl (meth)acrylate, less than about 1000, more preferably less than about 200, and most preferably less than about 60, parts per million by weight (ppmw) di(C1-C8 alkyl) ether and preferably less than about 500, more preferably less than about 50, ppmw C1-C8 alkyl acetate, with the sum of di(C1-C8 alkyl) ether, and C1-C8 alkyl acetate preferably being less than about 1500 ppm. More preferably, the recovered C1-C8 alkyl (meth)acrylate has a purity of at least about 99.5, more preferably at least about 99.8, and most preferably 99.9, weight percent C1-C8 alkyl (meth)acrylate and less than about 400, and most preferably less than about 300, ppmw of total di(C1-C8 alkyl) ether and C1-C8 alkyl acetate. At least a portion of the recovered heavies can be recycled to at least one esterification reaction zone or supplied to another reaction zone to crack the heavies and regenerate C1-C8 alcohol and (meth)acrylic acid.

### BRIEF DESCRIPTION OF THE DRAWINGS

FIG. 1 is a schematic depiction of a process for refining crude C1-C8 alkyl (meth)acrylate using a lights removal column (or splitter distillation column) in accordance with this invention.

FIG. 2 is a schematic depiction of a process for making crude C1-C8 alkyl (meth)acrylate from (meth)acrylic acid and C1-C8 alcohol and refining the crude C1-C8 alkyl (meth)acrylate using an extractor and lights removal column (or splitter distillation column) in accordance with this invention.

FIG. 3 is a schematic depiction of a process for making crude C1-C8 alkyl (meth)acrylate from (meth)acrylic acid and C1-C8 alcohol and refining the crude C1-C8 alkyl (meth)acrylate using a lights removal column (or splitter distillation column) in accordance with this invention.

### DETAILED DESCRIPTION

The esters of (meth)acrylic acid are typically manufactured by direct esterification of the alcohol with (meth)acrylic acid and employ a strong acid catalyst. The reaction step is equilibrium controlled and prompt removal of the reaction products is necessary to operate the reaction at an acceptable yield. With the (meth)acrylic esters of the lower molecular weight alcohols (C₁-C₄), this is typically accomplished by distillation whereby the reaction products and unreacted alcohol are removed from the top of a distillation tower associated with the reactor. For the esters of heavier alcohols, a tails process is employed where water is removed overhead in the reactor column and the product is removed from the base of the reactor.

The crude product from the reaction will predominately consist of unreacted alcohol, the product ester and water. Additionally several minor impurities may be present; dialkyl ether from dehydration of the alcohol and the esters of acidic impurities in the (meth)acrylic acid, such as acetic acid and propionic acid. Unreacted (meth)acrylic acid and the esterification catalyst may also be present. Substantial separation of the (meth)acrylate from these impurities is required prior to final refinement of the (meth)acrylate ester. The unreacted alcohol, the alkyl acetate and, for C₁-C₄ alcohols, the dialkyl ether are of lower boiling point than the product (meth)acrylate ester, and thus constitute the “light ends”. For (meth)acrylate esters from C₂ and C₈ alcohols, water is also a component of the “light ends”. The “light ends” are typically recycled back to the reactor or may undergo further processing. The product may be separated from these “light ends” via conventional distillation, typically performed under reduced pressure.

For alcohols containing less than 4 carbon atoms, an optional processing scheme employs extraction of the crude product with water to remove the majority of the alcohol and water of reaction from the crude product. A further refinement of the design might be the incorporation of an extraction solvent to improve the product recovery and reduce the alcohol breakthrough.

Whether produced via an overhead or tails process and whether preceded by an extractor or not, the feed to the splitter distillation column (or lights end column) will contain unreacted alcohol, the product ester, water, several minor impurities and, optionally, a solvent. The existence of numerous azeotropes among these components complicates the distillation scheme. Variations in the column feed composition may cause the column contents to shift from one azeotrope composition to another. The water solubility will vary in the different azeotropes possibly leading to the formation of a separate water region in the distillation column. Owing to their solubility in water, the (meth)acrylate esters, especially those of the C₁-C₄ alcohols, can partition into the aqueous phase. In the aqueous phase it is more difficult to inhibit the polymerization of the (meth)acrylate esters.

Operation of the splitter distillation column can be challenging owing to the propensity of the (meth)acrylate ester to polymerize. To mitigate this tendency, polymerization inhibitors are typically added. The most frequently used inhibitors are aromatic amines such as phenothiazine (PTZ) or alkyl substituted phenylenediames, and phenols such as hydroquinone (HQ). These materials may be used alone or in combination. These added inhibitors must be separated from the final commercial product and are typically expelled with the process waste stream. Process inhibitors add to the system costs and operational complexity. It is then very desirable to minimize the use of these in-process inhibitors.

Air addition has been shown to improve inhibitor efficacy of certain inhibitors, but its use is often complicated by flammability concerns. Also of issue is that the addition of gaseous components to the column can increase the amount of useful product and raw materials lost out the column vents thereby reducing unit efficiency and increasing costs. Condensation of these vapors via the use of heat exchangers can be problematic owing to freezing of the contained water in the “light ends” material.

Despite the presence of polymerization inhibitors and oxygen, fouling of the column trays by polymer can be experienced. We have unexpectedly found that through the addition of an alcohol or similarly water soluble solvent to the light ends removal column feed, in sufficient quantity as to avoid formation of water layers in the column, that the fouling can be avoided.

A proposed mechanism for the observed fouling is through the formation of a water phase in the column wherein the (meth)acrylate, but not the inhibitors may be soluble. The more water soluble phenolic inhibitors, such as hydroquinone, may be employed but they significantly partition into the organic phase requiring large dosages to adequately protect aqueous regions in process equipment. An alternate theory is that the alcohol acts as a simple diluent of the polymerizable monomer.

For alcohols containing more than 4 carbon atoms, their ability to solubilize water phases is much reduced, but they can still serve as a dilutent. With these higher molecular weight alcohols, a solvent of the appropriate boiling point and with greater water solubility can be utilized in place of the alcohol. In the following text, the term alcohol should be interpreted as an alcohol or a similar solvent which posses the appropriate boiling point for the process and sufficient water solubility as to aid in preventing the formation of water phases in the distillation tower.

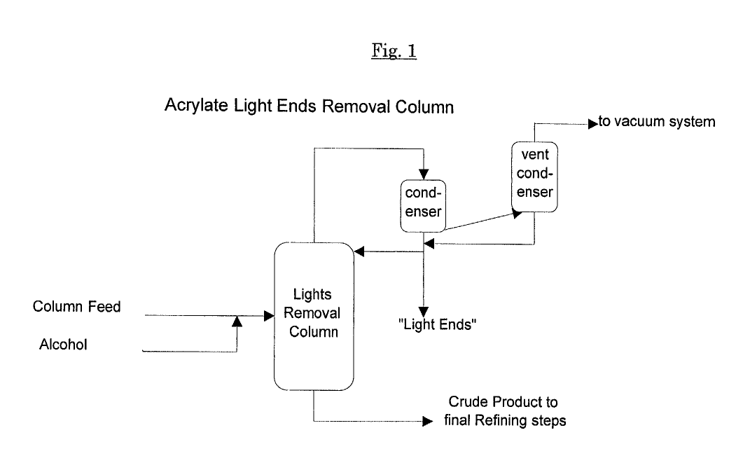

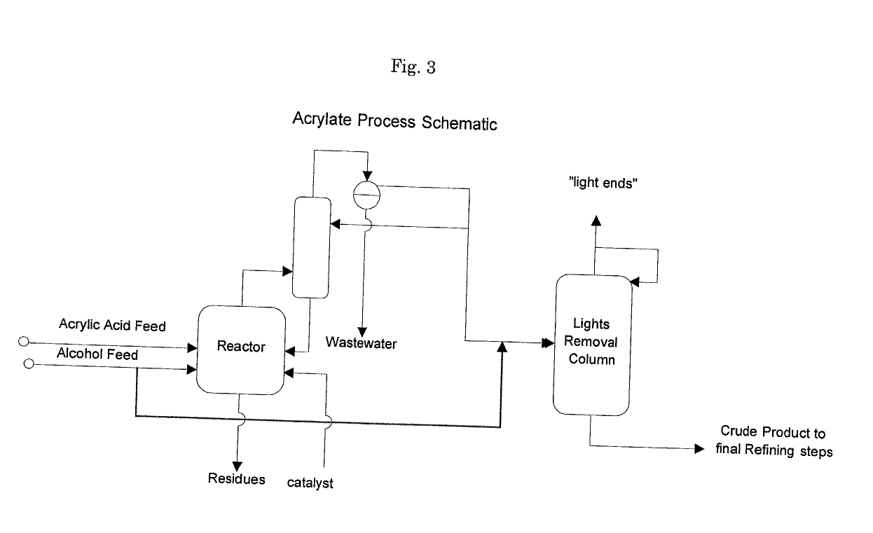

The addition of alcohol to the splitter distillation column feed is shown in FIG. 1 and as part of a process schematic in FIG. 3. The alcohol may be either “fresh” or “recycle” alcohol recovered in the manufacturing facility. The “light ends” recovered overhead may be recycled back to the reactor or subjected to further processing to recover the useful constituents. The column bottoms are further processed to yield the (meth)acrylate ester suitable for commercial applications, which typically involves further distillation under reduced pressure to remove heavy boiling impurities, such as the in-process inhibitors, and add the product storage inhibitor.

The amount of alcohol introduced into the column feed is an amount sufficient to avoid the formation of water layers in the column. Observation of the column overhead product maybe a good indication of when this condition is satisfied. The presence of insoluble water droplets in the column make suggest that in the column, larger water phases may be present which may lead to column fouling.

There are two advantages to feeding the alcohol to the column feed as opposed to simply increasing the alcohol ratio to the reactor, which would yield increased alcohol in the reactor overhead. In that the alcohol is water soluble, higher amounts of alcohol would be lost to the wastewater. Secondly a higher alcohol ratio in the reactor may increase the loss to reaction byproducts, i.e. ether and the Michael adduct of the alcohol and (meth)acrylate ester. Increasing the alcohol to acid ratio also unnecessarily consumes reactor space, thereby reducing reactor productivity.

An additional advantage to the invention is that it allows operation of the column's vent condenser at sub-zero (0°) temperatures without freezing. Freezing of the vent condenser from insoluble water, leads to operating the vent condenser at elevated temperatures which increases the losses to the vacuum system. Maintaining an elevated alcohol content in the column make avoids the formation of water layers and their freezing, thereby allowing operation of the vent condenser at lower temperatures, improving the recovery of product and raw materials.

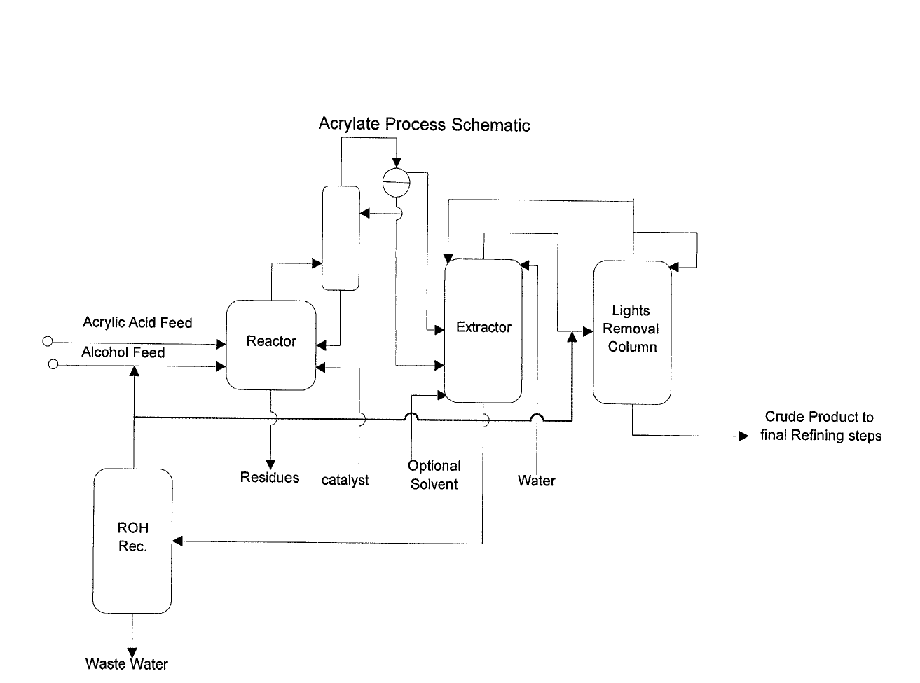

In FIG. 2, a process schematic employing an extractor between the reactor and light ends removal column (or splitter distillation column) is shown. Use of “recycle” alcohol to augment the light ends removal column feed is shown, but fresh alcohol could also be used. In this process it is still advantageous to feed the alcohol to the light ends removal column feed instead of to the reactor. The higher alcohol load on the extractor will increase the amount of water taken into the light ends removal column.

The invention is envisioned to be applicable to the manufacture of (meth)acrylic acid esters from C₁-C₈ alcohols. The product and unreacted alcohol being removed from the reactor and subjected to subsequent distillation, conducted at reduced or atmospheric pressure, to remove unreacted alcohol and light boiling impurities from the product, in the presence of polymerization inhibitors. The advantage of the invention being the avoidance of fouling in the distillation column. As used herein, the term “fouling” means the formation of oligomers of reactive monomers on the surface of equipment that prevents the equipment from operating the way in which it was intended.

Reduced inhibitor usage (don't need to add a water soluble inhibitor, i.e. hydroquinone or more costly nitroxyl compounds) Reduced fouling leading to improved process on stream time, reduced cleaning costs, and increased efficiencies as the vent condenser may be operated at a lower temperature thereby improving recovery of valuable product and raw material.

C1-C8 alkyl (meth)acrylates, e.g., ethyl acrylate, butyl acrylate, 2-ethylhexylacrylate and the like, are manufactured by the equilibrium-limited liquid phase reaction of C1-C8 alcohol, e.g., ethanol, butanol, 2-ethylhexanol and the like, and (meth)acrylic acid, e.g., acrylic acid. The reaction may be conducted as a batch process, but is preferably conducted as a continuous process in which the reactants and any adjuvants such as catalysts, inhibitors and solvents, are added periodically or uninterruptedly to, and products are removed periodically or uninterruptedly from, one or more reaction zones.

The reaction is typically conducted at temperatures within the range of about 70° to 170° C., more typically in the range of about 80° to 150° C. and most preferably in the range of about 90° to 140° C., but below a temperature that causes undue degradation of the reactants or C1-C8 alkyl (meth)acrylate and a temperature that causes the formation of polymer by reaction of the unsaturated moiety in (meth)acrylic acid.

The pressure at which the reaction is carried out also varies widely. Typically, reaction zone pressures range from subatmospheric to superatmospheric, e.g., from about 0.01 to 5 bar, more often from 0.1 to 2 bar, and most often from about 0.1 to 1.5 bar, absolute.

Catalysts appropriate for the reaction can be used and can be heterogeneous or homogeneous. Catalysts include acids such as sulfuric acid, sulfonic acids, and ion exchange resins having acidic functionality. The amount of catalyst can vary widely and often is in the range of about 0.001 to 20 weight percent of the liquid menstruum.

The reaction may be conducted in the presence of a solvent or, one or more of the reactants, products, coproducts and side reaction products may comprise the liquid media for the reaction. Where a solvent is used, it is preferably substantially inert under reaction conditions. Other adjuvants may be contained in the liquid reaction media, such as antioxidants, stabilizers, buffers, and the like.

The relative amounts of the reactants fed to the reaction zone may also vary widely and will often be selected based upon economic factors. Typically, reactants are supplied as fresh feed and recycle feed. Generally, the fresh feed C1-C8 alcohol and (meth)acrylic acid is supplied in an approximately stoichiometric ratio for producing the desired product, plus any additional amounts required to make up for losses due to side reactions. Often, the mole ratio of the C1-C8 alcohol to (meth)acrylic acid is between about 0.8:1 to about 1.2:1. Most preferably, as much as 90% of the fresh feed is consumed in the reaction zone. It should be understood that the amount of the reactants, and their relative concentrations, in the reaction zone may be different than that of the fresh feed due to recycling of unreacted reactants, with the mole ratio of C1-C8 alcohol to (meth)acrylic acid ranging from about 0.5:1 to about 5:1 and most preferably ranging from about 0.9:1 to about 1.5:1.

The esterification process of this invention is conducted in at least one liquid phase reaction zone. More than one reaction zone, or reaction stage, can be used. When more than one reaction zone is used, the reaction menstruum may be the same or different in each reaction zone.

In one embodiment of the invention, the conversion of the C1-C8 alcohol and (meth)acrylic acid to C1-C8 alkyl (meth)acrylate occurs in a single reactor. The C1-C8 alkyl (meth)acrylate produced in the reactor may be withdrawn as a liquid product stream from the reaction menstruum or removed as a gaseous product. If the product stream is withdrawn from the reaction menstruum as liquid, the conditions of the reaction zone should be such that the theoretical vapor-liquid equilibrium provides for at least about 50, preferably at least about 70 and more preferably at least about 80, percent of the C1-C8 alkyl (meth)acrylate contained in the reaction vessel to be in the liquid phase. The temperature and pressure should also be sufficient to drive the water to the gas phase so that gases removed from the reaction zone will be an azeotropic mixture. Often, under the conditions of the reaction zone including those promoting azeotrope formation, the vapor-liquid equilibrium for the reactants is such that at least about 50, preferably at least about 70, percent of each reactant contained in the reaction vessel is in the liquid phase. If it is elected to take the product stream as a gaseous effluent from the reaction zone, the conditions of the reaction zone should also be such that the theoretical vapor-liquid equilibrium provides for at least about 40, preferably at least about 50, and more preferably at least about 60 percent of the C1-C8 alkyl (meth)acrylate contained in the vessel to be in the vapor phase.

Regardless of how the product stream is taken from the reaction zone, it will contain C1-C8 alcohol, (meth)acrylic acid, C1-C8 alkyl (meth)acrylate and water and impurities such as heavies, di(C1-C8 alkyl)ether, and C1-C8 alkyl acetate.

In a single reactor process, azeotropic mixture produced in a liquid phase product production and removal regimen is removed from the reactor and typically subjected to liquid phase separation to remove the water, with at least a portion of the organic liquid comprising unreacted reactants being recycled to the reactor. Under gas phase product removal reaction conditions, the gaseous product stream removed from the reactor is subjected to distillation to return reactants to the reaction zone. The overhead from the distillation column is subjected to condensation and liquid phase separation to remove water, and a portion of the liquid is returned to the distillation column as reflux and the remaining organic phase is refined.

In another embodiment of the process, sequential reaction zones are employed. In this embodiment, liquid is withdrawn from a first reaction zone, which liquid contains product, co-product and unreacted reactants. While, in many instances, essentially all of the liquid withdrawn from the first reaction zone is passed to a second reaction zone, the broad concept contemplates using a portion of the liquid stream for other processing. In either case, additional reactant can be provided to the secondary reaction zone as a fresh feed or via a recycle stream. Also, an intervening separation step may be used to separate product and/or coproduct from the liquid. The separation may simply be a liquid phase separation to remove, e.g., water via a flashing or distillation unit operation, or coproduct or product separation via a membrane separation or a sorption process.

Sequential reactor zones provide process flexibility. For instance, no overhead stream need be removed from the first reaction zone. Thus, a plug flow reactor could be used if desired. Generally, the residence time of the liquid menstruum in the primary reaction zone is sufficient to produce C1-C8 alkyl (meth)acrylate at a concentration to within 50, typically, within about 70, and sometimes at least about 90 or 95, percent of the theoretical equilibrium concentration of C1-C8 alkyl (meth)acrylate in the reaction menstruum under the conditions of the reaction (for given reactant concentrations). Because no overhead stream need be taken, savings in equipment and energy can be achieved. The reaction can proceed further in the second reaction zone to achieve the desired amount of conversion. Advantageously, at least about 50, preferably at least about 70, and most preferably between about 75 and 90, percent of the total amount of C1-C8 alkyl (meth)acrylate produced in the process is produced in the first reaction zone. Often, the first reaction zone is operated such that an amount equivalent to at least about 50, preferably at least about 70, and most preferably between about 75 and 90, percent of the fresh feed of at least one, most preferably both, of the reactants is consumed in the primary reaction zone.

The conditions of the second reaction zone are maintained such that the C1-C8 alkyl (meth)acrylate product is vaporized. The conditions may include a temperature and pressure such that the product flashes into the gaseous phase. Preferably, under the conditions of the second reaction zone including azeotrope formation, the vapor-liquid equilibrium for C1-C8 alkyl (meth)acrylate is such that less than about 50, preferably less than about 30, percent of C1-C8 alkyl (meth)acrylate contained in the reaction vessel is in the liquid phase. Also, under the conditions of the second reaction zone including azeotrope formation, the vapor-liquid equilibrium for C1-C8 alcohol and (meth)acrylic acid preferably is such that less than about 50, and in some instances less than about 30, percent of at least one of the reactants contained in the reaction vessel is in the liquid phase.

Often the second reaction zone is at temperatures within the range of about 80° to 170° C., more typically within the range of about 90° to 150° C., but below a temperature that causes undue degradation of the reactants or C1-C8 alkyl (meth)acrylate. Preferably, the temperature of the liquid menstruum in the second reaction zone is equal to or greater, e.g., 0° to about 15° C. greater, than the temperature of the menstruum in the first reaction zone. The pressure in the second reaction zone can also vary widely. Typically, pressures range from subatmospheric to superatmospheric, e.g., from about 0.01 to 5 bar, most often from about 0.1 to 1.5 bar, absolute. Often, the pressure in the second reaction zone is equal to or below the pressure in the first reaction zone, e.g., about 0 to 0.9 bar below the pressure in the first reaction zone.

The reaction in the second reaction zone is conducted in the presence of liquid comprising at least one of C1-C8 alcohol, (meth)acrylic acid, heavies, and one other liquid component, e.g., a solvent. The reaction menstruum may be the same or different in the first reaction zone and in the second reaction zone. Where a solvent is used, it is preferably substantially inert and is substantially non-volatile under reaction conditions. Even though C1-C8 alcohol and (meth)acrylic acid will be vaporized, the reactants are maintained in the liquid of the second reaction zone for a time sufficient to produce C1-C8 alkyl (meth)acrylate. A gas is withdrawn from the second reactor and comprises C1-C8 alcohol, (meth)acrylic acid, C1-C8 alkyl (meth)acrylate, water and impurities such as heavies, di(C1-C8 alkyl) ether and C1-C8 alkyl acetate.

Catalysts appropriate for the equilibrium reaction can be used in the second reaction zone and may be the same or different catalyst used in the first reaction zone. Advantageously, if the catalyst in the first reaction zone is a homogeneous catalyst or a slurried heterogeneous catalyst, it can be passed with the liquid to the second reaction zone. Alternatively, the use of a heterogeneous, stationary catalyst in the first reaction zone, enables a different catalyst to be used in the second reaction zone.

Heavies can be formed by a Michael reaction. The heavies product (including dimer) is typically an equilibrium product. The heavies may comprise a substantial portion of the liquid menstruum; for instance, at least about 10, more typically, about 20 to 90 or more, weight percent of the menstruum. In such an embodiment, (meth)acrylic acid and C1-C8 alcohol can be recovered as a bottoms fraction, and at least a portion of the bottoms fraction can be recycled to at least one of the first or second reaction zones. Advantageously, the second reaction zone can be operated under conditions favorable to cracking the heavies.

In accordance with this invention, the crude C1-C8 alkyl (meth)acrylate stream withdrawn from the reaction zone and typically containing C1-C8 alkyl acetate, C1-C8 alcohol, water and heavies is refined by distillation in a splitter distillation column which produces an overhead fraction containing C1-C8 alkyl acetate and C1-C8 alcohol and a bottoms fraction containing C1-C8 alkyl (meth)acrylate, heavies, and preferably less than about 1000 ppmw of total C1-C8 alkyl acetate.

The actual compositional make-up of the reaction zone product stream depends on a number of factors. For example, the composition of the crude C1-C8 alkyl (meth)acrylate stream depends, in part, upon the composition of the reactants. Impurities in the feeds will also influence the composition; for instance, acetic acid is often an impurity in (meth)acrylic acid and C1-C8 alkyl acetate will be generated in the reaction zone. Advantageously, if desired, the processes of this invention enable a mixed acetic acid and (meth)acrylic acid feed to be used to co-generate C1-C8 alkyl acetate and C1-C8 alkyl (meth)acrylate, both valuable products. Further, whether the crude stream is recovered as a gaseous effluent affects composition. Moreover, catalysts and other adjuvants used also affect the composition. The crude stream will generally contain a total of at least about 0.05 weight percent of total C1-C8 alkyl acetate and di(C1-C8 alkyl) ether, and often will contain at least about 0.1, and up to about 55, weight percent of total C1-C8 alkyl acetate and di(C1-C8 alkyl) ether.

If water is present to form azeotropes, as is usually and preferably the case, the crude stream will actually be comprised of a wide variety of constituents including several azeotropes containing one or more of the above-mentioned components.

The number of theoretical plates in the splitter distillation column will vary depending upon the composition of the crude stream and the desired concentration of the C1-C8 alkyl (meth)acrylate in the bottoms stream. Generally, the splitter column will have at least about 5 or 10 theoretical plates, more typically, between about 10 and 20 theoretical plates. The number of theoretical plates is preferably sufficient to provide a bottoms fraction containing less than 1500, preferably less than about 500, ppmw of total C1-C8 alkyl acetate. The point of feed to the splitter column will also depend upon the crude stream composition. For streams supplied directly from the esterification reaction zone, the feed to the splitter column is frequently at a point that permits at least about 5 theoretical plates to exist below the point of feed. The splitter column may be of any suitable design, including trays, packing or a combination of trays and packing. The splitter column is generally operated with a reflux ratio of between about 1 and 50. The reflux ratio is generally selected based upon the composition of the crude stream and the desired separation.

The temperature and pressure of the splitter distillation column will also depend upon the sought separation. Often, the splitter column is operated at subatmospheric pressure in order that the separation can be effected at temperatures below those that can result in degradation or side reactions of any of the components in the feed stream. The pressure at the base of the splitter column typically ranges from about 0.01 to 1.1, preferably from about 0.1 to 0.8, bar absolute. The temperature at the base of the splitter column is generally within the range of about 80° to 150° C., preferably 90° to 130° C. The pressure at the top of the splitter column typically is between 0.01 and 0.8, typically, 0.05 to 0.6, bar below the pressure at the base of the splitter column. The temperature at the top of the splitter column is generally about 40° to 110° C., preferably 50° to 100° C.

A water soluble organic solvent is introduced into the splitter distillation column in an amount sufficient to prevent the formation of one or more water layers in the column. Mixtures of one or more different solvents may be employed if desired. Suitable water soluble organic solvents include, for example, alcohols, e.g., ethanol, propanol, butanol, 2-ethylhexanol and the like, diols, triols, polyols, glycol ethers, e.g., 2-ethoxyethanol and 2-methoxyethanol, nitrites, lactones, pyrrolidones, formamides, sulfoxides and the like. Illustrative solvents useful in this invention include, for example, propionitrile, 1,3-dioxolane, 3-methoxypropionitrile, N-methylpyrrolidone, N,N-dimethylformamide, 2-methyl-2-oxazoline, adiponitrile, acetonitrile, epsilon caprolactone, glutaronitrile, 3-methyl-2-oxazolidinone, dimethyl sulfoxide and sulfolane. The amount of solvent employed is not critical to the subject invention and need only be that amount sufficient to prevent or minimize fouling in the splitter distillation column. In general, the amount of solvent employed may range from about 1 percent by weight or less up to about 25 percent by weight or more based on the total weight of the column feed.

The overhead fraction is subjected to a further distillation in a C1-C8 alcohol recovery column. Advantageously, the recovered C1-C8 alcohol is sufficiently free from di(C1-C8 alkyl) ether and C1-C8 alkyl acetate that it is desirably recycled to an esterification reaction zone. The C1-C8 alcohol recovery column is operated in the presence of water to form azeotropes such that C1-C8 alcohol is recovered as a bottoms stream. The amount of water present should be sufficient such that at least about 70, most preferably essentially 100, percent of the total C1-C8 alkyl acetate and di(C1-C8 alkyl) ether forms an azeotrope. The number of theoretical plates in the C1-C8 alcohol recovery column will vary depending upon the composition of the portion of the overhead stream from the splitter column that is passed to the C1-C8 alcohol recovery column and the desired purity of the C1-C8 alcohol. Generally, the alcohol recovery column will have at least about 5 theoretical plates, preferably, between about 7 and 20 theoretical plates. The number of theoretical plates is preferably sufficient to provide a bottoms fraction containing less than about 5000, more preferably less than about 3000, ppmw of total di(C1-C8 alkyl) ether and C1-C8 alkyl acetate. Frequently, from 2 to about 16 theoretical plates are provided below the point of the feed to the C1-C8 alcohol recovery column. The C1-C8 alcohol recovery column may be of any suitable design, including trays, packing or a combination of trays and packing. The C1-C8 alcohol recovery column is generally operated with a reflux ratio of between about 1 and 60. The temperature and pressure of the column will depend upon the sought separation. Often, the column is operated at subatmospheric pressure in order that the separation can be effected at temperatures below those that can result in degradation or side reactions of any of the components in the feed stream. The pressure at the base of the column typically ranges from about 0.01 to 1.1, preferably from about 0.1 to 0.8, bar absolute. The temperature at the base of the alcohol recovery column is generally within the range of about 50° to 120° C., preferably 60° to 100° C.

The total C1-C8 alkyl acetate and di(C1-C8 alkyl) ether in the overhead fraction from the C1-C8 alcohol recovery column is often at least about 5 and can comprise up to about 80, weight percent of the overhead fraction. Since water is used in the distillation process, the overhead from the C1-C8 alcohol recovery column is often subjected to phase separation. The organic phase can be used to provide reflux and the aqueous fraction discarded. Alternatively, at least a portion of the aqueous fraction may be recycled to the C1-C8 alcohol recovery column to promote the desired azeotrope formation.

The bottoms fraction from the splitter distillation column contains C1-C8 alkyl (meth)acrylate, C5-C8 dialkyl ether and heavies. This bottom fraction may be acceptable as is or may be further processed in a C1-C8 alkyl (meth)acrylate separation column to remove the heavies. The separation may be a flash separation in which a C1-C8 alkyl (meth)acrylate stream is taken overhead, or may be a distillation. Generally, the C1-C8 alkyl (meth)acrylate separation column will have at least 2 theoretical plates, typically 3 to about 10 theoretical plates. The C1-C8 alkyl (meth)acrylate separation column may be of any suitable design, including trays, packing or a combination of trays and packing. The C1-C8 alkyl (meth)acrylate separation column is generally operated with a reflux ratio of between about 0.05 and 10. The temperature and pressure of the column will depend upon the sought separation. Often, the separation column is operated at subatmospheric pressure in order that the separation can be effected at temperatures below those that can result in degradation or side reactions of any of the components in the feed stream. The pressure at the base of the separation column typically ranges from about 0.01 to 1.1, preferably from about 0.02 to 0.8, bar absolute. The temperature at the base of the C1-C8 alkyl (meth)acrylate column is generally within the range of about 60° to 170° C., preferably 70° to 150° C.

In an advantageous aspect of the invention, at least a portion of the bottoms fraction from the C1-C8 alkyl (meth)acrylate separation column is passed to a reactor operating under cracking conditions to recover (meth)acrylic acid and C1-C8 alcohol. As previously disclosed, the processes that use sequential reactors can be operated such that the second reaction zone is at a temperature and contains heavies in sufficient concentration such that cracking of the heavies is facilitated.

Although the foregoing discussion has referenced a splitter distillation column in combination with a C1-C8 alcohol recovery column and a C1-C8 alkyl (meth)acrylate separations column, this invention contemplates the use of a single splitter distillation column with a plurality of reflux and off take locations to accomplish the separations effected by the recovery and separation columns.

Employing the process of the present invention, a C1-C8 alkyl (meth)acrylate product can be produced having the preferred composition as described herein. (Meth)acrylates with such low levels of lights, especially di(C1-C8 alkyl) ether, exhibit significantly reduced odor as compared to commercially available (meth)acrylates and enable the production of unexpectedly high quality product from lower quality feedstock. These improvements are evident in polymers made from the C1-C8 alkyl (meth)acrylate, making the C1-C8 alkyl (meth)acrylate more desirable for demanding applications such as low odor, low VOC coatings and adhesives. This combination of improved quality and lower production cost has not been heretofore appreciated.

The following examples are intended to illustrate one or more, but not all, embodiments within the scope of the present invention.

In both examples the feed to the distillation and column operating parameters were held the same except as noted below.

### EXAMPLE 1

An ethyl (meth)acrylate distillation column was operated under vacuum with phenothiazine and hydroquionone added to the overhead of the column. The column feed, also contained phenothiazine and entered near the middle of the tray section of the column. Air was introduced into the column base at a rate sufficient to mitigate polymerization in the calandria. The vent condenser was maintained at the lowest possible temperature as to avoid freezing, ˜+5° C. The average resulting composition was (concentrations in per cent by weight)

| | Ethyl | Ethyl | Ethyl | |
|---|---|---|---|---|
| Ethanol | Acetate | (meth)acrylate | Ether | Water |
| 5.82 | 27.82 | 54.87 | 7.30 | 4.33 |

The column make (and reflux) additionally contained 150-200 ppm of phenothiazine.

Operating in this fashion, water droplets were occasionally seen in the column overhead samples. The column trays fouled with polymer three times in a 16 month period requiring costly unit downtime and cleaning. Losses to the vent system were significant based on engineering calculations.

### EXAMPLE 2

The column was operated as in example # 1 except ethanol was added to the column feed in sufficient quantity as to avoid the formation of a water droplets in the overhead composition. (concentrations in per cent by weight)

| | Ethyl | Ethyl | Ethyl | |
|---|---|---|---|---|
| Ethanol | Acetate | (meth)acrylate | Ether | Water |
| 13.18 | 30.54 | 40.76 | 9.24 | 6.68 |

The column make (and reflux) contained 150-200 ppm of phenothiazine. The column operated in excess of 9 months with no fouling of the trays as evidenced by no increase in differential pressure across the column trays and no degradation of separation.

The higher ethanol content prevented freezing of the vent condenser, despite the higher water content, allowing the vent condenser to be operated at a significantly colder temperature, approximately −5 to −15° C., thereby improving recovery of materials. This is evidenced by the increased concentration of ethyl ether compared to the composition shown in Example 1.

Although the invention has been illustrated by the preceding example, it is not to be construed as being limited thereby; but rather, the invention encompasses the generic area as hereinbefore disclosed. Various modifications and embodiments can be made without departing from the spirit and scope thereof.

1. A process for refining a C1-C8 alkyl (meth)acrylate-containing stream comprising C1-C8 alkyl (meth)acrylate, C1-C8 alkyl acetate, heavies and C1-C8 alcohol, the process comprising the steps of:
a. introducing said stream into a splitter distillation column to provide an overhead fraction comprising C1-C8 alkyl acetate, C1-C8 alkyl (meth)acrylate and C1-C8 alcohol and a bottoms fraction comprising C1-C8 alkyl (meth)acrylate and heavies;
b. introducing a second stream comprising a water soluble organic solvent into said splitter distillation column in an amount sufficient to prevent or minimize fouling in said splitter distillation column;
c. withdrawing from the splitter distillation column the bottoms fraction; and
d. separating heavies from said bottom fraction.

2. The process of claim 1 wherein step d is accomplished by introducing the bottoms fraction into a C1-C8 alkyl (meth)acrylate distillation column to provide an overhead product containing C1-C8 alkyl (meth)acrylate and a bottom product containing heavies, and withdrawing from the C1-C8 alkyl (meth)acrylate distillation column the overhead product.

3. The process of claim 1 further comprising the steps of:
e. withdrawing from the splitter distillation column the overhead fraction; and
f. introducing the overhead fraction into a C1-C8 alcohol recovery distillation column to provide an overhead stream comprising C1-C8 alkyl acetate and a bottoms stream comprising C1-C8 alcohol; and
g. withdrawing from the C1-C8 alcohol recovery distillation column the bottoms stream.

4. The process of claim 1, wherein the stream introduced into the splitter column further comprises water.

5. The process of claim 4, wherein the overhead fraction from the splitter distillation column further comprises azeotropes of at least one of di(C1-C8 alkyl) ether and C1-C8 alkyl acetate.

6. A process for refining a (meth)acrylate-containing stream comprising (meth)acrylate, ether, acetate, heavies and alcohol, the process comprising the steps of:
a. introducing said stream into a splitter distillation column to provide an overhead fraction comprising ether, acetate, (meth)acrylate and alcohol and a bottoms fraction comprising (meth)acrylate and heavies;
b. introducing a second stream comprising a water soluble organic solvent into said splitter distillation column in an amount sufficient to prevent or minimize fouling in said splitter distillation column;
c. withdrawing from the splitter distillation column the bottoms fraction; and
d. separating heavies from said bottom fractions.

7. A process for the production of C1-C8 alkyl (meth)acrylate comprising the steps of:
a. reacting in at least one reaction zone an (meth)acrylic acid-containing feedstock with a C1-C8 alcohol-containing feedstock to produce a C1-C8 alkyl (meth)acrylate-containing product stream comprising C1-C8 alkyl (meth)acrylate, C1-C8 alkyl acetate, heavies and C1-C8 alcohol;
b. introducing said product stream into a splitter distillation column to provide an overhead fraction comprising C1-C8 alkyl acetate, C1-C8 alkyl (meth)acrylate and C1-C8 alcohol and a bottoms fraction comprising C1-C8 alkyl (meth)acrylate and heavies;
c. introducing a second stream comprising a water soluble organic solvent into said splitter distillation column in an amount sufficient to prevent or minimize fouling in said splitter distillation column;
d. withdrawing from the splitter distillation column the bottoms fraction;
e. separating heavies from said bottom fraction;
f. withdrawing from the splitter distillation column the overhead fraction; and
g. introducing the overhead fraction into a C1-C8 alcohol recovery distillation column to provide an overhead stream comprising C1-C8 alkyl acetate and a bottoms stream comprising C1-C8 alcohol;
h. withdrawing from the C1-C8 alcohol recovery distillation column the bottoms stream; and
i. supplying at least a portion of the bottom stream to said at least one reaction zone.

8. The process of claim 7 wherein water is supplied at least initially to said at least one reactor zone.

9. The process of claim 7 wherein the reaction of the feedstocks occurs in the presence of a homogeneous or heterogeneous catalyst.

10. The process of claim 7 which further comprises the production of C1-C8 alkyl acetate.

11. The process of claim 1 wherein said C1-C8 alkyl (meth)acrylate-containing stream and said second stream are combined prior to introducing into said splitter distillation column.

12. The process of claim 1 wherein said second stream comprises an alcohol.
